(19) United States
(12) Patent Application Publication
(10) Pub. No.: US 2015/0327789 A1
(43) Pub. Date: Nov. 19, 2015

(54) Electrode

(71) Applicant: SMARTBRAIN AS, Oslo (NO)

(72) Inventor: Njaal Haldor Sjaaheim, Oslo (NO)

(21) Appl. No.: 14/649,928

(22) PCT Filed: Dec. 5, 2013
(86) PCT No.: PCT/EP2013/075703

§ 371 Date: Jun. 5, 2015

Related U.S. Application Data
(60) Provisional application No. 61/733,446, filed on Dec. 5, 2012.

(30) Foreign Application Priority Data
Dec. 5, 2012 (EP) 12195714.6

(51) Int. Cl. A61B 5/0478; A61B 5/0408; A61B 5/0492; A61N 1/04

(52) U.S. Cl. CPC A61B 5/0478; A61N 1/0404; A61B 5/0408; A61B 5/0492; A61N 1/0456; A61B 2562/14

(57) ABSTRACT

The present invention concerns an electrode for measuring a bio-potential on, and/or providing electric stimulation to, a skin surface of a subject, said electrode comprising a device for providing a conductive fluid, the device comprises at least one container (1) for storing a conductive fluid (2), said container comprises at least one controllable outlet (3), wherein the conductive fluid (2) is retainable in the container at an above ambient pressure, during use said pressure is sufficient to provide a jet (8) of conductive fluid through the outlet (3).

### TECHNICAL FIELD

The present invention concerns the field of wet electrodes, more specifically the provision of a wet electrode having a device for delivery of electro-conductive fluids.

### BACKGROUND OF THE INVENTION

When choosing an electrode system in for instance electroencephalogram (EEG) measurements, there is a compromise between the time used for application, i.e. the time it takes to apply and prepare the electrodes onto the scalp of a subject, and the quality of the obtained measurements. The preferred electrodes are the so called wet electrodes, since these provide the best signal to noise ratio. The reason for the difficulties in obtaining a high quality signal is the low amplitude (μV) of the measured bio-potentials. By using wet electrodes, the electrode-skin impedance is lowered by using an electro-conductive fluid to obtain a better electrical contact between the electrode and skin. The contact is further depending on many different variables such as; the amount of dead skin cells (abrasion level), individual hair structure, length, volume, density, humidity and salinity level. In the physical recording there is also electrostatic discharge noise from electrode settling, electrochemical reactions between the electrode material, fluids and subjects biology. Due to these variables, and the required application of a conductive fluid, the application of wet electrodes is a highly time-consuming process compared to dry electrodes. It is estimated that a normal 64-channel EEG-montage requires one hour of skilled labour. The current “state of the art” connection-quality for both dry and wet electrodes is heavily dependent on the operator skills and technique. Even if two different montages seem similar at a glance, they can behave unpredictably because of differences in gel volume, surface area, dispersion and electrode-skin pressure. This problem can arise between individual electrodes in one subject, due to movement and uneven drying of the gel. This also poses a problem when the same subject is recorded in multiple sessions, making the recording of bio-potential more unpredictable than necessary.

The current state of the art commonly consists of applying the required electro-conductive fluid, for instance by using a syringe with blunted needle, followed by a manual adjustment of each individual electrode to lower the impedance (preferable below 10k ohm), between the skin and each electrode site. The manual adjustments are necessarily time-consuming because the process of hair and dead skin cell removal are in most subjects needed to obtain low impedances. Access to each electrode site, after applying the electrodes, is also required in order to accommodate the application of the conductive fluid. Simple access is not always possible.

Patent application US 2011/0288604 A1 discloses a system for the automatic release of a conductive fluid in connection with a defibrillator. The fluid is contained in a receptacle and is being released by igniting a gas cartridge which causes a dose of fluid to burst.

The goal of the present invention is to alleviate or avoid at least some of the disadvantages of the present wet electrode techniques.

### SUMMARY OF THE INVENTION

The present invention provides an electrode having a device comprising a container within which a conductive fluid is stored or contained. The electrode is suitable for incorporation into for instance an EEG-cap, or similar, used in an EEG/TES system, used in the recording of bio-potentials. The electrode can also be fitted with other fixing arrangements, as self-adhesive plasters for Electrocardiography (ECG), Electromyography (EMG) or other bio-potential applications. The fluid in the device is kept at a pressure above ambient atmospheric pressure. Said pressure is preferably high enough to allow for a full discharge of the conductive fluid. The container comprises at least one outlet in fluid contact with the conductive fluid. When not in use, the outlet is in a closed state preventing the conductive fluid from exiting the container. The outlet may comprise any suitable means for regulating the flow of conductive fluid through a passage from the interior of the container to the exterior of the container. Such means may comprise any suitable type of valve, membrane, plug or similar, which is able to close the outlet until release of the fluid is desired. Said means are made up of, or are in combination with, any suitable actuator and/or transducer. When desired, the conductive fluid is released from the container at a suitable pressure, the pressure of the fluid is preferably sufficient to remove the stratum corneum, i.e. the outermost layer of the epidermis consisting of dead skin cells, in the same process. The dispersion pattern of the released fluid could be made in a way that provide a consistent penetration level and covered surface area regardless of hair density- and volume. The device of the electrode may therefore provide a predictable and repeatable skin-electrode connection with equal dispersion and volume of gel in all recording sites.

The present invention provides the possibility of acquiring bio-potential's, faster and easier without compromising signal quality. This is especially important with multiple electrode Electroencephalogram (EEG) recordings and the application of Trans-cranial Electric Stimulation (TES) or High Definition focal-targeted Trans-cranial Electric Stimulation (HD-TES. This technique requires stable skin-electrode impedance and a predictable conductive surface area for electrical stimulation. The present innovation is not limited by the type of non-invasive electrodes and is adaptable to all bio-potential recording or stimulation where an easy and reliable skin-electrode connection is beneficiary. The future of digital EEG and TES is promising, but the standardization and elimination of variables in electrode montages is critical for cross-validation, usability and competitiveness.

The present invention is further defined in the following and the attached claims:

In one embodiment, the invention provides an electrode having a device for providing conductive fluid, the device comprises at least one container for storing a conductive fluid, said container comprises at least one controllable outlet, wherein the conductive fluid is retainable in the container at an above ambient pressure and the electrode is preferably adapted to be integrated in a system for measuring a bio-potential on, and/or providing electric stimulation to, a skin surface of a subject. Depending on the system for which the electrode is to be adapted, the container and/or outlet may be designed in any suitable shape or form, and may comprise any suitable connecting means, to make the device integrable in the system. The required shape, form or connecting means, will vary depending on the specific system to be used, but will easily be inferred by a skilled person based on common knowledge and routine experiments. The electrode is intended to be integrated into the system during use.

The system for measuring a bio-potential is preferably suitable for EEG, ECG, EMG, TES and/or HD-TES.

In some embodiments of the invention, the interior of the container is divided in a first and a second section by a membrane, the first section containing a propellant fluid and the second section able to contain the conductive fluid, the conductive fluid being in fluid contact with at least one outlet.

In some embodiments of the invention, the interior of the container is divided in a first and a second section by a piston, the first section containing a propellant fluid and the second section able to contain the conductive fluid, the conductive fluid being in fluid contact with at least one outlet.

In some embodiments of the invention, the container, when in use, comprises an elastic membrane in contact with the conductive fluid, said conductive fluid pressurized by the compressive forces of said membrane. In these cases the device of the electrode according to the invention does not require a propellant fluid to pressurize the conductive fluid. When the container does not store any conductive fluid, the membrane is not elastically extended. When the container is being filled with conductive fluid the elastic membrane will extend and exert a compressive force on the fluid. The fluid will in this manner be kept pressurized until the outlet is opened during use. The elastic membrane may constitute an integral part of the container, or divide an interior of the container in two sections. In the latter case, the container will comprise a first section with the conductive fluid in fluid contact with the outlet, and a second section. The second section may comprise a compressed gas if the second section is not in fluid contact with the surroundings. The gas of the second section may be pressurized before the conductive fluid is filled into the first section, or it may be pressurized due to the expansion of the first section.

In some embodiments of the invention, the propellant fluid is a compressed gas.

In some embodiments of the invention, the outlet described above is a remote controlled valve.

In a preferred embodiment of the electrode according to the invention, the pressure of the conductive fluid is sufficient to provide a flow, or jet, of conductive fluid through the outlet, said flow, or jet, able to remove at least parts of the stratum corneum.

In an embodiment of the electrode according to the invention, the device comprises multiple containers. Each container will then contain a separate amount of conductive fluid in contact with a corresponding outlet.

In some embodiments according to the present invention, the electrode is adapted to be integrated into a membrane, an electrode cap or an electrode pad. Said membrane, electrode cap, or pad, is a part of a system for measuring a bio-potential on, and/or providing electric stimulation to, a skin surface of a subject.

Further, the present invention comprises the use of an electrode, as described above, in a system for measuring a bio-potential on, and/or providing electric stimulation to, a skin surface of a subject.

Further, the present invention provides a system for measuring a bio-potential, wherein said system comprises an electrode according to the invention.

In a final embodiment, the present invention provides a method for measuring a bio-potential on, and/or providing electric stimulation to, a skin surface of a subject, comprising the following step:
applying a conductive fluid to the skin surface at a velocity or pressure sufficient to remove at least parts of the stratum corneum.

The method for measuring a bio-potential on, and/or providing electric stimulation to, a skin surface of a subject may also comprise at least one of the following steps:
applying at least one electrode according to the invention close to the skin surface; and
measuring the bio-potential on, or providing electric stimulation to, the skin surface.

The regulating means may comprise any suitable type of transducer, actuator or combination of both, that may release the conductive fluid instantly or controllably. The fluid regulating actuator, for instance in a valve, may work with any force and the interaction between them, for instance; electrical, mechanical, electromagnetic (including light), chemical, acoustic, thermal or similar in any combination with any type of external or internal energy source. A transducer could be used to convert targeted energy to power and activate the actuator for instance by converting differential pressure, wave energy, or similar. The regulating means or actuator could optionally be remote controlled, or activated for instance in response to a signal from an electrode, or a remote transmitter could send wireless signal to a transducer picking up targeted energy and thus working as a local actuator and energy source for the actuator. When used in the present description, the term “valve” is intended to comprise any type of assembly suitable for regulating the fluid flow through the outlet of the container. Such valves may preferably be a common electromechanical valve, but other types may also be advantageous in various settings or applications, such as a Piezoelectric type valve, a thermal release type valve wherein a thermo sensitive valve or decomposition of a material is actuated by a heating coil or similar, a chemical type valve where a reaction releases the pressure, a mechanical valve that is actuated by differential pressure or have an upper pressure limit actuated by additional pressure to the reservoir, or a resonant material that break up by an external signal having an appropriate wavelength.

### SHORT DESCRIPTION OF THE DRAWINGS

FIG. 1 shows a schematic cross-section of an embodiment of a device suitable for the electrode according to the invention.

FIG. 2 shows a schematic cross-section of a device suitable for the electrode according to the invention.

FIG. 3 shows a schematic cross-section of a device suitable for the electrode according to the invention.

FIG. 4 shows a perspective view of the device in FIG. 3.

FIG. 5 shows a further embodiment of a device suitable for the electrode according to the invention.

FIG. 6 shows the embodiment in FIG. 5.

FIG. 7 shows a schematic drawing of electrodes according to the invention incorporated into a membrane for qEEG recordings.

### DETAILED DESCRIPTION OF THE DRAWINGS

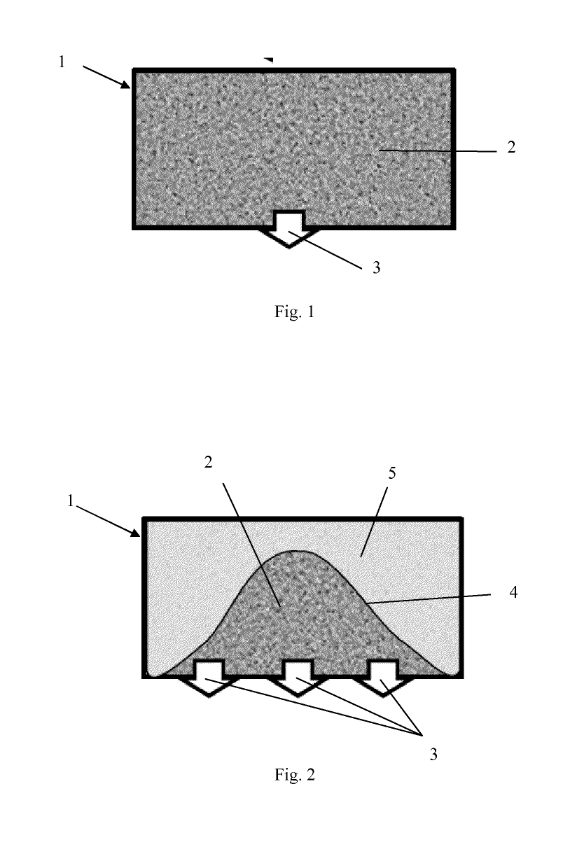

FIGS. 1-6 shows various embodiments of a device for providing conductive fluid in the electrode according to the invention. The electrode element itself (i.e. the element of the electrode performing the measuring of a bio-potential or providing an electric stimulation) is preferably arranged close to the outlet (3) of the device. In some instances the electrode element may be integrated into the outlet, and in others arranged in the vicinity of the outlet. The only requirement regarding the arrangement/position of the electrode part is that it is close enough to the outlet to obtain a good electrical contact between the electrode part and the skin, i.e. the electrode part is preferably in contact with both the skin and the conductive fluid, optionally in electrical contact with the skin via the conductive fluid. The electrode part is further connected to a measuring equipment/apparatus, or optionally an electric stimulation providing equipment/apparatus. Means for connecting the electrode to such equipment include wireless connections, direct connections by wire etc., and are well known to the skilled person. As is also well known to the skilled person, the electrode element is preferably made of tin, brass, gold, or silver, and preferably of silver chloride.

A schematic drawing of a device for providing conductive fluid 2 is shown in FIG. 1.

The device is suitable for incorporation into for instance a membrane, or skull cap, in a system for the recording of bio-potentials. The device comprises a fluid-proof container 1 enclosing the conductive fluid. The conductive fluid is kept at a pressure above ambient, i.e. above the atmospheric pressure. The container comprises an outlet 3. The outlet 3 is able to control or regulate the flow of conductive fluid 2 from the interior of the container to its exterior. The outlet may comprise any appropriate type of controllable valve, membrane or similar, often in connection with a nozzle. The flow through the outlet may be controlled by any suitable means, for instance by a connected electric signal or a remote radio signal. The pressure of the conductive fluid is preferably high enough to cause the fluid to be ejected through the outlet as a jet or flow sufficient to abrade the skin such that the layer of dead skin cells (the stratum corneum) is removed, optionally at least parts of said layer is removed. The pressure required of the conductive fluid, when stored in the container, to obtain such an abrasive effect is dependent on the type of outlet used. In the embodiment shown in FIG. 1, the pressure of the conductive fluid is obtained by for instance formulating the fluid together with an appropriate propellant and a gelator.

In the device shown in FIG. 2, the interior of the container 1 is divided into two compartments. One compartment contains a suitable propellant 5, such as a compressed gas, and the other compartment contains a conductive fluid 2. The two compartments are separated by a membrane 4. The membrane is fluid-proof and flexible. The propellant pressurizes the conductive fluid via the membrane 4. As shown in the specific embodiment of FIG. 2, the membrane 4 need not be elastic. In this particular embodiment, the device has three separate outlets 3. The number of outlets may be varied according to need. The different outlets may be regulated separately or simultaneously, i.e. each outlet may be opened and/or closed independent of the other outlets, or not. In a further embodiment similar to the one shown in FIG. 2, the membrane is elastic. In the case of an elastic membrane, the device does not require a propellant. The introduction of conductive fluid will in this case extend the membrane to accommodate the fluid volume and as a consequence the membrane will pressurize the fluid due to the compressive forces applied from the membrane on the fluid.

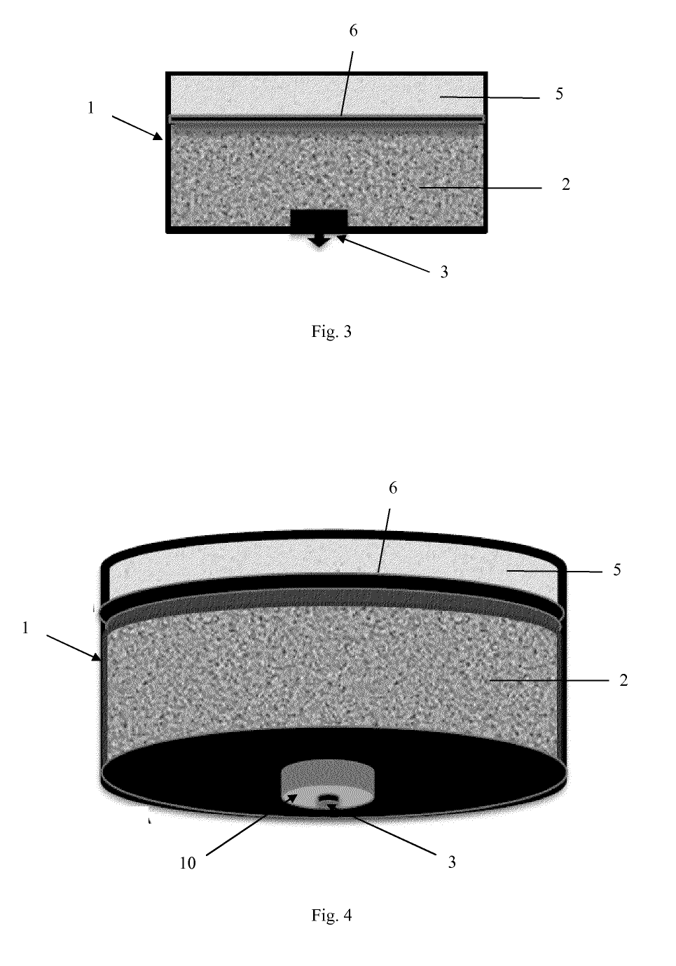

An embodiment of a device for an electrode according to the invention is shown in FIG. 3. In this embodiment, the container 1 is divided into two compartments. One compartment contains a suitable propellant 5, such as a compressed gas, and the other compartment contains a conductive fluid 2. The two compartments are separated by a fluid-proof piston 6. The propellant pressurizes the conductive fluid via the piston 6.

A perspective view of the device in FIG. 3 comprising an electrode element 10 is shown in FIG. 4. In this embodiment of an electrode according to the invention the electrode element surrounds the outlet 3.

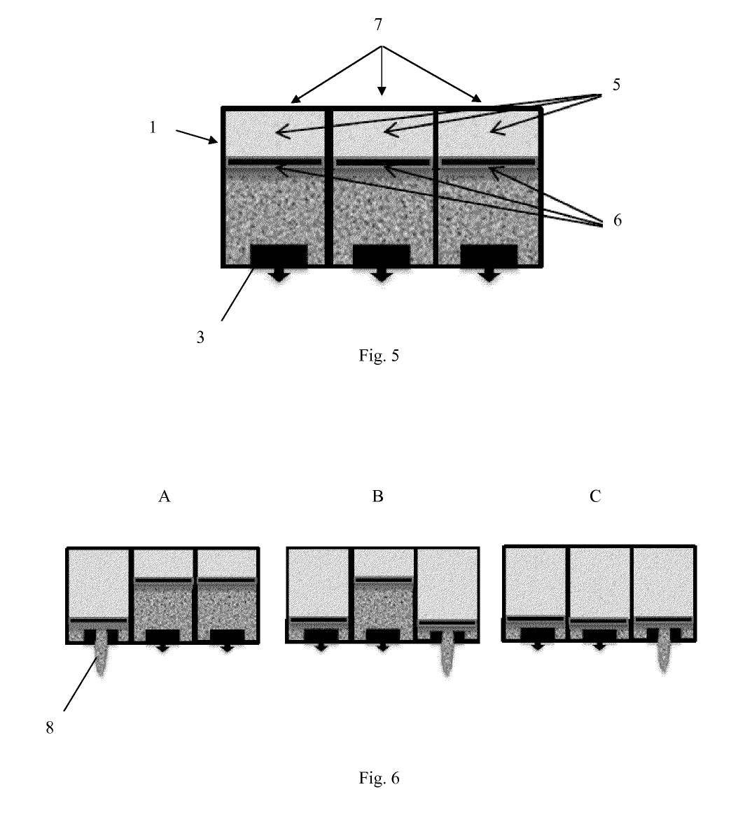

FIG. 5 shows a device comprising three containers, or described in another way; a device wherein the container 1 comprises three separate chambers 7, each chamber 7 comprising two compartments separated by a piston 6. Each pair of separated compartments consists of one compartment containing a propellant 5 and one compartment containing a conductive fluid. Each chamber has an outlet in fluid contact with the conductive fluid. The various outlets may be controlled independently of each other. In FIG. 6, the device in FIG. 5 is shown in three different stages A, B and C. A first outlet is opened in stage A, causing the formation of a jet 8 of conductive fluid. If the achieved bio-potential is too weak, stages B and C may be initiated to obtain lower skin-electrode impedance.

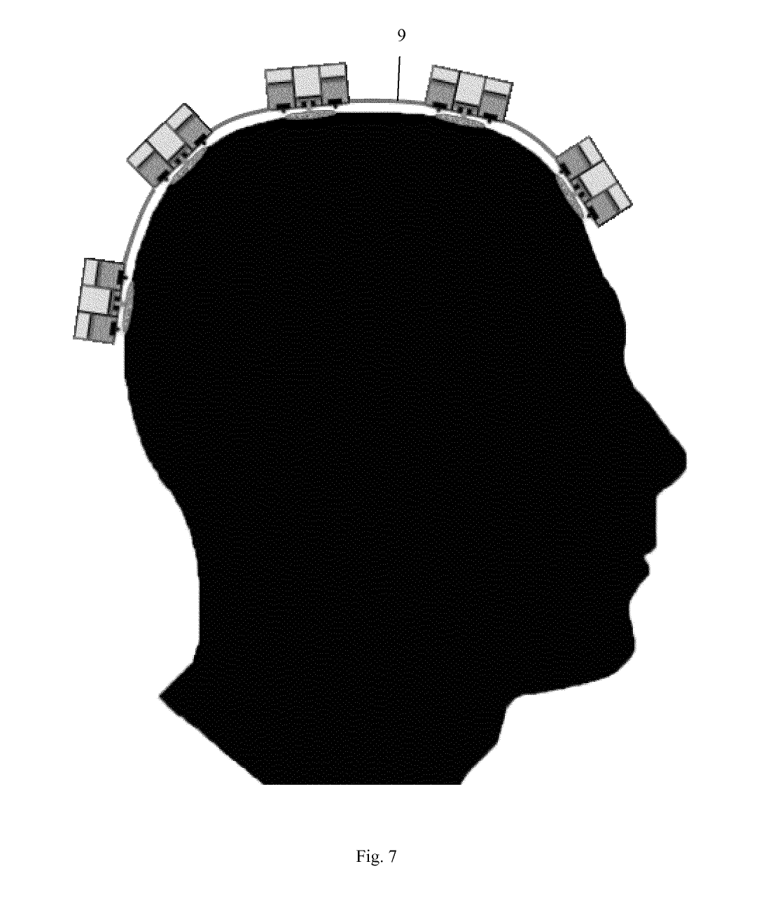

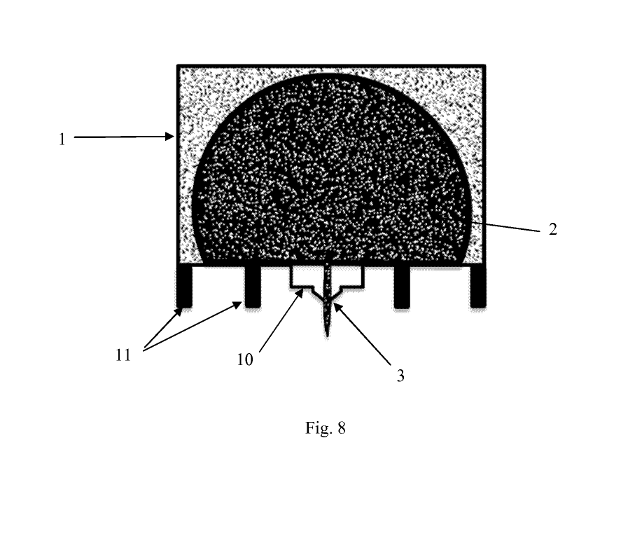

As described above, the electrode according to the invention is primarily intended to be incorporated into a system for measuring a bio-potential, for instance a qEEG (quantitative EEG) recording system. A schematic drawing of such a system is shown in FIG. 5. The device according to the invention is here incorporated into the membrane 9 of a cap arranged on the skull of a subject, is shown in FIG. 7.

A further embodiment of an electrode according to the invention is shown in FIG. 8. As shown in FIG. 4, the electrode element 10 is formed around, or being part of, the outlet 3. Distance elements 11 are arranged on the electrode to ensure that the outlet 3, and/or the electrode element, is at a given distance from a skin surface when the electrode is applied thereto. Although not required, such distance elements may help in obtaining more reproducible measurements, for instance by ensuring a more uniform emplacement when multiple electrodes are used.

The electrode according to the invention may dispense conductive fluid in a single burst, in multiple bursts over time or any other advantageous schedule.

The fluid pressure is advantageously set to provide a flow-/jet-velocity able to abrade at least parts of the stratum corneum. Adequate flow-/jet-velocity of the fluid will contribute to a good bio-potential signal independent of possible obstacles such as dirt and hair.

The conductive fluid may be any fluid able to conduct electric signals compatible to the intended use. Such fluids comprise for instance a saline liquid or gel. To enhance the abrasive effect of the fluid, it may also comprise small abrasive particles, such as various types of crystals.

The container of the device of the electrode may advantageously be made in a conductive material, and being a part of, or constituting, the element of the electrode being in contact with the skin of a subject.

The container may be of any design which makes it suitable to be integrated into a system for measuring a bio-potential. In addition to the membrane or skull cap, described above with reference to FIG. 7, other suitable systems for integration may be for instance electrode pads.

1-17. (canceled)

18. An electrode for measuring a bio-potential on a skin surface of a subject, said electrode comprising a device for providing a conductive fluid, the device comprises at least one container (1) for storing a conductive fluid (2), said container comprises at least one controllable outlet (3), wherein the conductive fluid (2) is retainable in the container at an above ambient pressure, during use said pressure is sufficient to provide a jet (8) of conductive fluid through the outlet (3), wherein the outlet (3) comprises a mechanical valve that is actuated by differential pressure.

19. An electrode according to claim 18, wherein the valve has an upper pressure limit actuated by additional pressure to the conductive fluid.

20. An electrode according to any of claims 18, wherein the interior of the container is divided in a first and a second section by a fluid-proof movable element (4, 6), during use the first section contains a pressurized propellant fluid (5), and the second section contains the conductive fluid (2) which is in fluid contact with the at least one outlet (3).

21. An electrode according claim 18, wherein the interior of the container is divided in a first and a second section by a membrane (4), or a piston (6), the first section able to contain a propellant fluid (5), and the second section able to contain the conductive fluid (2) and being in fluid contact with the at least one outlet (3).

22. An electrode according to claim 18, wherein the container comprises an elastic membrane able to pressurize a retained conductive fluid, the fluid being in fluid contact with the at least one outlet (3), when the membrane is extended.

23. An electrode according to claim 22, wherein the membrane is an integrated part of the container.

24. An electrode according to any of claim 23, wherein the propellant fluid (5) is a compressed gas.

25. An electrode according to claim 18, wherein the outlet (3) is a remote controlled valve.

26. An electrode according to claim 18, wherein the container is adapted to be integrated into a layer, such as a membrane, an electrode cap or an electrode pad.

27. An electrode according to claim 26, wherein the electrode is replaceable.

28. An electrode according claim 18, comprising at least one distance element (11) arranged such that a desired predetermined distance is achieved between a skin surface and the outlet (3), and/or an electrode element (10), when the device is placed on said skin surface, preferably the electrode comprises multiple distance elements, in the form of rods, arranged around the outlet.
